(19) United States
(12) Patent Application Publication
(10) Pub. No.: US 2006/0211987 A1
(43) Pub. Date: Sep. 21, 2006

(54) Vein stabilizer devices and methods of using same

(76) Inventor: Arthur Myles Williams, Decatur, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/192,626

(22) Filed: Jul. 29, 2005

Related U.S. Application Data
(60) Provisional application No. 60/662,594, filed on Mar. 18, 2005.

(51) Int. Cl. A61M 5/00; A61F 11/00; A61B 17/00

(52) U.S. Cl. 604/116; 606/203; 606/108

(57) ABSTRACT

Medical devices such as vein stabilizer devices and intravenous starter kits and methods of using the vein stabilizer devices are disclosed herein. One exemplary starter kit includes a vein stabilizer device and a tourniquet.

### CLAIM OF PRIORITY

This utility application claims the benefit of and priority to U.S. provisional application entitled, “Vein Stabilizer and Stopcock” having Ser. No. 60/662,594, filed Mar. 18, 2005, which is incorporated herein by reference in its entirety.

### FIELD OF THE INVENTION

The present disclosure relates to vein stabilizer devices and tourniquets and kits embodying the same.

### BACKGROUND

Veinal blood flows only towards the heart. Within the veins are leaflet valves which allow blood flow in this direction and stop its reverse flow. When inserting a needle into the vein, typically a tourniquet is positioned on an extremity such as an arm or leg. The veinal blood flow is thereby halted or constricted and the veins on the distal (hand or foot) side of the tourniquet begin to swell or distend becoming quite defined and noticeable. Once the desired vein for penetration has been selected, the operator pierces the vein with the needle tip.

The problems encountered with veinal penetration are widely known: the vein may be missed altogether; the needle may completely penetrate through the entire vein; and unsuccessful penetration efforts may injure or damage the vein or surrounding tissue—all to the pain and injury of the patient. After a tourniquet has been applied on an arm, the veins in the extremity become somewhat inflexible. The veins however, still remain measurably resilient to thwart even an experienced operator's attempt at needle insertion. Essentially, any exerted pressure by a needle tip that is not longitudinally aligned with a distended vein may cause the vein to move and resist a piercing attempt.

In the past, known efforts at successful veinal penetration have dealt with using a form of a tourniquet about the arm or leg and some form of an immobilizing means about the vein to be pierced. Alternatively, the operator may attempt to hold the vein between two fingers striving to keep it stationary and thus avoid painful and injurious false efforts in piercing the vein.

Various bulky and cumbersome instruments have also been utilized in attempting to immobilize the vein for longitudinal needle insertion. Types of vein stabilizers, retainers, or immobilizers, such as a hand-held vein stabilizer for placing over a vein, have helped prevent transverse movement of the vein but manifest various limitations in their ease of operation, release, and success. Moreover, many vein stabilizers leave the individual administering the needle vulnerable to needle sticks.

More than 8 million healthcare workers in the United States work in hospitals or other healthcare settings. According to a publication released by the U.S. Centers for Disease Control (CDC) in November 1999 (“Preventing Needlestick Injuries in Health Care Settings”), estimates indicate that about 600,000 to 800,000 needlestick injuries occur annually. Data suggest that at an average hospital, workers incur approximately 30 needlestick injuries per 100beds per year.

The emotional impact of a needlestick injury can be severe and long lasting, even when a serious infection is not transmitted. This impact is particularly severe when the injury involves exposure to the HIV virus or other viruses. Stress reactions requiring counseling have also been reported. Not knowing the infection status of the source patient can accentuate the healthcare worker' stress. In addition to the exposed healthcare worker, colleagues and family members may suffer emotionally.

The CDC recommended that for selecting and evaluating needle devices with safety features, the following criteria could be used: (a) the safety feature works effectively and reliably; (b) the device is acceptable to the healthcare worker; and (c) the device does not adversely affect patient care.

It would be advantageous to develop vein stabilizers, immobilizers, or retainers that will not only aid a healthcare worker in more efficiently accessing a patient's vein with a needle, but will also serve to reduce the risk of needlestick injuries.

### SUMMARY

Briefly described, embodiments of this disclosure include intravenous line starter kits. One exemplary intravenous line starter kit includes a vein stabilizer device, wherein the vein stabilizer device includes an enclosure with an aperture therethrough, the aperture configured to slidably receive a tourniquet, and a first set of prongs fixedly extending from a first edge of the enclosure, the first set of prongs forming a first groove therebetween, the first groove configured to receive a needle; and a tourniquet designed to be threaded through the aperture of the enclosure.

Other embodiments of this disclosure include vein stabilizer devices. One exemplary vein stabilizer device includes a stopcock that includes an enclosure with an aperture therethrough, the aperture configured to receive a tourniquet; a first set of prongs fixedly extending from a first edge of the housing, the first set of prongs forming a first notch therebetween, the first notch configured to receive a needle; and a second set of prongs extending from a second edge of the housing, the second set of prongs fixedly forming a second notch therebetween, the second notch configured to receive the needle, and wherein the second notch is of a different size than the first notch.

Other embodiments of the disclosure provide for methods of using a vein stabilizer device. One exemplary method includes the following steps: (a) threading a tourniquet through a vein stabilizer device, the vein stabilizer device comprising a housing with an aperture therethrough, the aperture configured to slidably receive the tourniquet; (b) tightening a tourniquet around a patient's arm, thereby distending at least one vein; (c) selecting the distended vein for puncture after application of the tourniquet and vein stabilizer; (d) engaging a portion of skin covering the pre-selected vein with the vein stabilizer device, the vein stabilizer device further comprising a first set of prongs extending from a first edge of the housing, the first set of prongs forming a first groove therebetween, the first groove configured to stabilize a needle and a vein; (e) stabilizing the distended vein is stabilized between the grooves; and (f) puncturing the vein with a needle.

### BRIEF DESCRIPTION OF THE DRAWINGS

Many aspects of this disclosure can be better understood with reference to the following drawings. The components in the drawings are not necessarily to scale. Moreover, in the drawings, like reference numerals designate corresponding parts throughout the several views.

FIG. 1 illustrates a perspective view of an embodiment of the disclosed vein stabilizer stopcock.

FIG. 2 illustrates a perspective view of an embodiment of the disclosed vein stabilizer stopcock.

FIG. 3 illustrates a front view of an embodiment of the disclosed vein stabilizer stopcock.

FIG. 4 is a rear view of an embodiment of the disclosed vein stabilizer stopcock.

FIG. 5 is a side view of an embodiment of the disclosed vein stabilizer stopcock.

FIG. 6 is a top view of an embodiment of the disclosed vein stabilizer stopcock.

FIG. 7 depicts partial top views of various embodiments of a groove or notch in the disclosed vein stabilizer stopcock. Other profiles are possible.

FIG. 8 illustrates a perspective view of an embodiment of the disclosed vein stabilizer apparatus.

FIG. 9 illustrates a perspective view of an embodiment of the disclosed vein stabilizer stopcock.

FIG. 10 illustrates a perspective view of an embodiment of the disclosed vein stabilizer apparatus in operation on a patient.

FIG. 11 illustrates a perspective view of an embodiment of the disclosed vein stabilizer apparatus in operation on a patient.

FIG. 12 illustrates various top views of an embodiment of the disclosed vein stabilizer stopcock, demonstrating how the vein stabilizer stopcock can be oriented at various angles on a tourniquet.

FIG. 13 illustrates a perspective view of an embodiment of the disclosed vein stabilizer stopcock.

### DETAILED DESCRIPTION

Disclosed are intravenous starter kits, vein stabilizer devices that employ a stopcock, and methods of using the disclosed vein stabilizer devices. Generally, one such vein stabilizer device includes a stopcock. The stopcock is configured to allow a tourniquet to be threaded through the stopcock.

As used herein, the term “stopcock” generally refers to a device or mechanism that can be pressed against an outer skin of a patient over a vein location such that by intermittently rotating the stopcock up or pressing the stopcock against the skin, the flow of blood through the vein can be substantially controlled. By engaging the stopcock against the skin tightly, blood flow can be substantially stopped through the vein, thereby causing the vein to become distended and enabling it to be subsequently punctured with a needle. By rotating or pivoting the stopcock away from the skin, blood is allowed to flow through the vein and through the needle for collection. In addition to substantially controlling the flow of blood through the vein, the stopcock also can serve to stabilize the vein so that a needle can be inserted into the vein. Thus, the stopcock has at least one of two functions: controlling flow of blood and/or stabilizing the vein for puncture.

The stopcock can be made of a material that is able to be sterilized (e.g., through autoclaving, ethylene oxide gas, and radiation) and reused, and/or made of a material that is disposable. For example, the stopcock can be made of a resin (e.g., acrylic, a polystyrene, polycarbonate), a wood, and/or a metal (e.g., stainless steel, aluminum, alloys). In addition, the stopcock can be made of a clear or colored transparent plastic. When the stopcock is made of a resin, it can be made, for example, by injection molding. In one embodiment, the stopcock is of a size that can fit in an intravenous line starter pack, yet is large enough to allow a tourniquet to be snugly threaded through it.

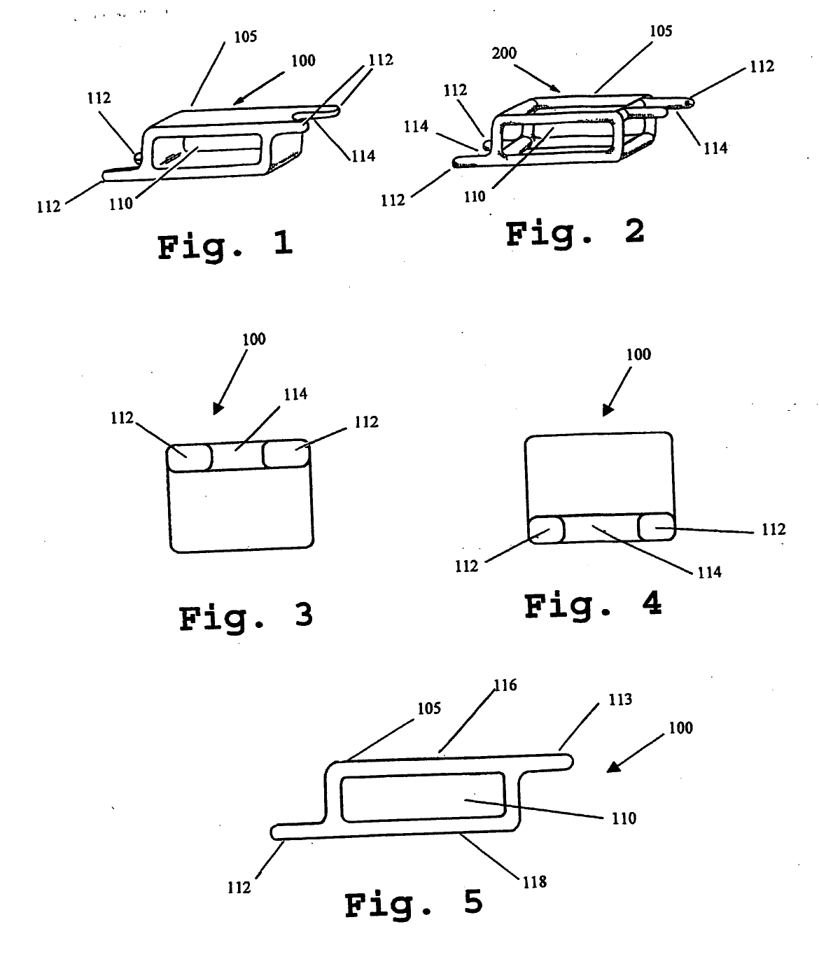

Having generally described the vein stabilizer device, reference is made to the drawings. As seen in FIG.1, the vein stabilizer stopcock 100 can be configured as a small housing or enclosure 105 with an enclosed aperture 110 formed therethrough. Although the stopcock 100 is shown with the enclosure 105 having a substantially rectangular shape in FIG. 1, the enclosure 105 may be in the form of other shapes, including for example, substantially circular, semicircular, substantially ovoid, semi-ovoid, or elliptical. The aperture 110 is generally of a size and shape to slidably receive a tourniquet (see, e.g., FIGS. 8-10).

One edge of the enclosure 105 has two fingers or prongs 112 extending from it that forms a notch or groove 114 between the prongs 112. In one embodiment, the prongs 112 fixedly extend from an edge of the enclosure 105 such that they are not moveable. The stopcock can have an aperture 110 formed therein of a configuration different from the exact configuration shown in FIG. 1. For example, depicted in FIG. 2 is an embodiment of a stopcock 200 with the enclosure 105 having the aperture 110 extending therethrough in two planes, e.g., a plane parallel to the prongs 112, and a plane perpendicular to the prongs 112.

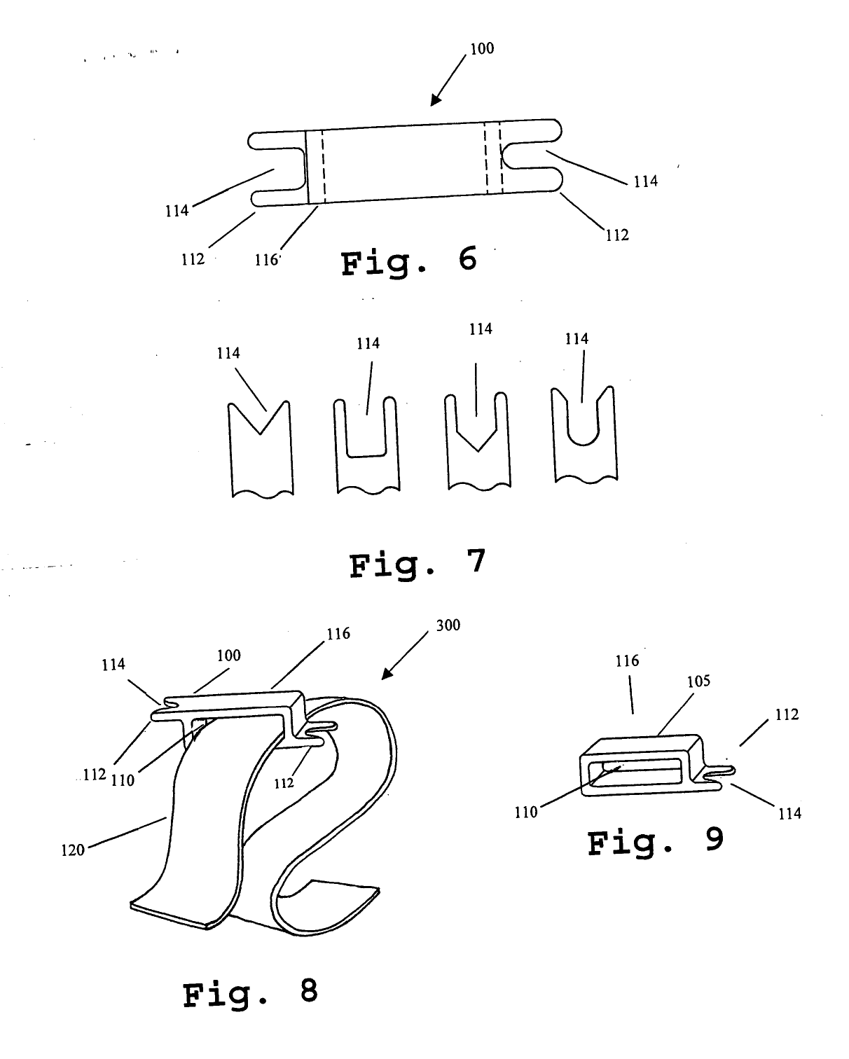

FIGS. 3-4 illustrate the stopcock 100 from the front and rear views, respectively. FIGS. 34 illustrate that the prongs 112 extend substantially straight out from the edge of the stopcock 100, with the groove 114 formed between the two prongs 112. FIG. 6 is a top view of the stopcock 100.

FIG. 5 is a side view of the stopcock 100. The stopcock 100 has a top surface 116 and a bottom surface 118 with the prongs 112 extending from each surface. During use, either the top surface 116 or the bottom surface 118 engage the patient's skin. FIG. 5 illustrates that, in one embodiment of the stopcock 100, the two prongs 112 that extend from the enclosure 105 can be continuations of the top surface 116 and/or the bottom surface 118. In one embodiment (shown in FIG. 5), prongs 112, 113 extend in opposite directions from each other. In other words, with the length of the enclosure 105 running from left to right, the first set of prongs 112 extend to the left from either the top surface 116 or the bottom surface 118, and a second set of prongs 113 extend from the right and from the top surface 116 or the bottom surface 118.

The grooves 114 formed from the first set of prongs 112 and the second set of prongs 113 can be of different shapes and/or sizes. For example, with respect to shape, the grooves 114 can be substantially “v” shaped, substantially “u” shaped, or substantially rectangular in shape, as shown in FIG. 7. With respect to size, the prongs 112 and 113 can be spaced further apart in one set than the other, or one set of prongs can be thicker in width than the other, such that the grooves 114 can be of different widths. In this manner, a user of the stopcock 100 can determine which size or shape is appropriate for use depending on the size of the vein and/or needle, and “flip” the stopcock 100 over with the tourniquet remaining threaded therethrough. Thus, the grooves 114 can allow for stabilization of different sized veins.

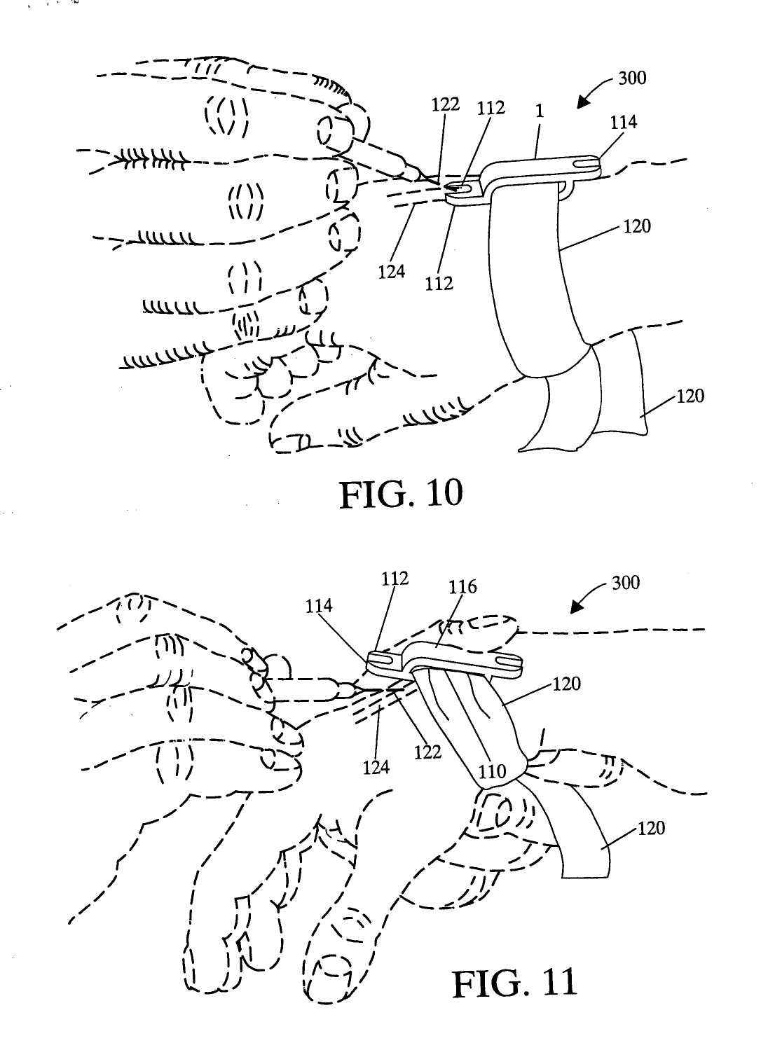

FIGS. 9 and 13 depict embodiments of the stopcock. In FIG. 9, only one set of prongs 112 extend from the enclosure 105. In FIG. 10, the stopcock 400 is generally of a circular or semi-circular shape. In FIG. 13, the aperture can either be of a size to accommodate tubular-type tourniquets and/or of a size to accommodate a user's finger (e.g., the thumb).

As indicated above, FIG. 8 depicts an embodiment of the vein stabilizer device 300. The device 300 includes a tourniquet 120 threaded through the aperture 110 of the stopcock 100. FIGS. 10 and 11 show the vein stabilizer device 300 including the tourniquet 120 in use on a patient during a venapuncture procedure. The tourniquet can also be configured to slidably receive other accessories for the venaunture procedure (not shown). For example, the tourniquet can be designed to have attached thereto or slid thereon a vial holder, gauze, bandages or bandage holder, surgical tape, scissors, etc.

The disclosure also relates to intravenous line starter kits or “IV start kits.”In one embodiment, the IV start kit includes the stopcock, the tourniquet, and a needle.

One method of using the vein stabilizer device 300 includes threading the tourniquet 120 through a vein stabilizer device 300, in a manner shown in FIG. 8. The tourniquet 120 is then tightened around a patient's arm, wrist, or hand, thereby distending at least one vein 124, as shown in FIG. 10. Typically, if more than one vein is distended, then the vein 124 that most distended is selected for puncture. In some cases, only one vein is distended.

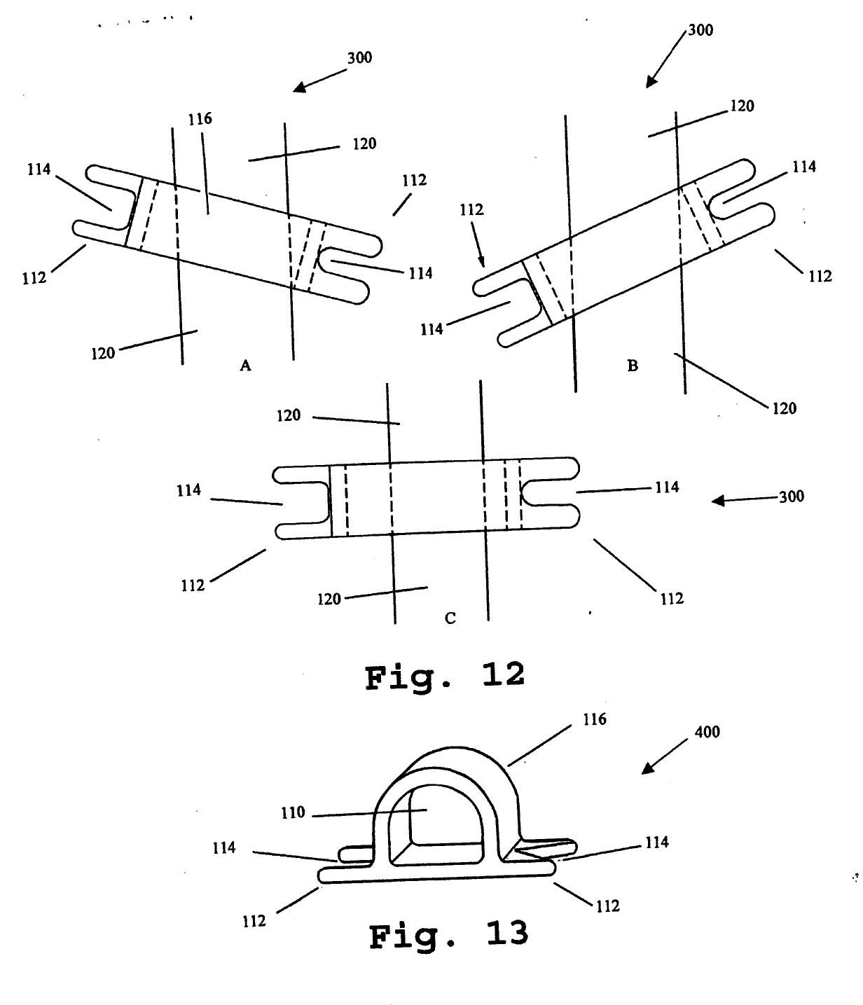

The user of the vein stabilizer device 300 then engages a portion of skin covering the pre-selected vein 124 with the vein stabilizer device 300, such that the distended vein 124 is stabilized within the groove 114 of the stopcock 100, shown in FIG. 10. FIG. 12 illustrates how an embodiment of a vein stabilizer device 300 can be rotated with the tourniquet 120 disposed thereon to precisely select and stabilize the vein to be punctured. Veins do not always run perfectly straight along a patient's hand or arm. Therefore, the stopcock 100 of the device 300 can be rotated (with the tourniquet 120 already threaded therethrough) in a plane that is parallel to the patient's arm, as shown in exemplary embodiments A, B, and C. The vein 124 is therefore disposed and stabilized in the groove 114. After the vein is stabilized, the user of the device 300 then punctures the vein 124 with a needle 122 (FIGS. 10 and 11).

In one embodiment of the method, after puncture, the user can pivot one edge of the stopcock 100 upwardly by depressing an edge of the top surface 116 of the stopcock 100, thereby releasing a flow of blood into the vein 124, as shown in FIG. 11. This action can also be described as rocking back the leading edge of the stopcock 100 that is adjacent the needle 122 to expose more of the vein 124 being punctured. The stopcock 100 can be pressed and released at one end, e.g., by prongs 113, such that the prongs 113 act as a handle to start and stop the flow of blood into or out of the needle 122. In this way, the stopcock 100 can control the flow of blood if, for example, multiple vials of blood are desired to the collected from the patient.

In addition or alternatively, after puncture, the tourniquet 120 can be loosened and removed from the patient while the needle 124 is still inserted in the patient. In one embodiment of the method, the tourniquet 120 is removed while leaving both the stopcock 100 and the needle 124 in place.

In one embodiment of the disclosed method of using the vein stabilizer device 300, the needle 122 is inserted at an angle to the patient's skin, and longitudinal to the vein 124, as shown in FIG. 10. In particular, it should be noted that in the embodiment of the method demonstrated in FIG. 10, both the user's hands are distal to the tip of the needle 122, e.g., one hand is holding a barrel of the needle while the other hand is disposed out of the way, such as holding the patient's hand. Neither of the user's hands are in danger of an accidental needle stick in the embodiment shown in FIG. 10. It is only after the needle has been safely inserted that the user need pivot the stopcock 100 upwardly, as shown in FIG. 11. Upon pivoting the stopcock 100 upwardly, the needle 122 can then be inserted further into the vein 124. In the case that the needle 122 is used to start an IV line, the stopcock 100 and tourniquet 120 can be easily removed without disturbing or knocking out the IV line.

Many variations and modifications may be made to the above-described embodiments of the vein stabilizer devices, the stopcocks, the intravenous line starter kits, and the methods of using the vein stabilizer devices vein. All such modifications and variations are intended to be included herein within the scope of this disclosure and protected by the following claims.

The following is claimed:

1. An intravenous line starter kit, the kit comprising:
a vein stabilizer comprising
an enclosure with an aperture therethrough, the aperture configured to receive a tourniquet, and
a first set of prongs fixedly extending from a first edge of the enclosure, the first set of prongs forming a first groove therebetween, the first groove configured to receive a needle; and
a tourniquet designed to be threaded through the aperture of the enclosure.

2. The device of claim 1, wherein the aperture is configured to slidably receive the tourniquet in a snug fit, wherein the fit is configured to enable the enclosure to slide up and down the tourniquet.

3. The device of claim 1, wherein the aperture is configured to slidably receive the tourniquet in a snug fit, wherein the fit is configured to enable the stopcock to rotate in a plane parallel to a patient's arm at an angle up to about 90°.

4. The device of claim 1, wherein the enclosure further comprises a second set of prongs extending from a second edge of the enclosure, the second set of prongs fixedly forming a second groove therebetween, the second groove configured to receive a needle, and wherein the second groove is of a different size than the first groove.

5. The device of claim 1, wherein the aperture is of a shape chosen from at least one of: substantially rectangular, substantially circular, and substantially elliptical.

6. The device of claim 1, wherein the tourniquet is of a shape chosen from at least one of: substantially tubular and substantially flat.

7. The device of claim 1, wherein the vein stabilizer is configured to substantially stop the flow of blood in a vein when the vein stabilizer is disposed against a patient's skin covering the vein, and is configured to allow the flow of blood in the vein when it is removed from being disposed against the patient's skin covering the vein.

8. The device of claim 1, wherein the enclosure is made of a material that can be sterilized with an autoclave.

9. The device of claim 1, wherein the enclosure is disposable.

10. The device of claim 1, wherein the aperture is configured to slidably receive the tourniquet in a snug fit, but wherein the fit is configured to enable the enclosure to rotate in a plane perpendicular to a patient's arm at an angle up to about 90°.

11. A vein stabilizer device, comprising:
a stopcock comprising
an enclosure with an aperture therethrough, the aperture configured to slidably receive a tourniquet;
a first set of prongs fixedly extending from a first edge of the housing, the first set of prongs forming a first notch therebetween, the first notch configured to receive a needle; and
a second set of prongs extending from a second edge of the housing, the second set of prongs fixedly forming a second notch therebetween, the second notch configured to receive the needle, and wherein the second notch is of a different size than the first notch.

12. The device of claim 11, wherein the aperture is configured to slidably receive a tourniquet in a snug fit, but wherein the fit is configured to enable the stopcock to rotate in a plane perpendicular to a patient's arm at an angle up to about 90°.

13. The device of claim 11, wherein the aperture is configured to slidably receive a tourniquet in a snug fit, but wherein the fit is configured to enable the stopcock to rotate in a plane parallel to a patient's arm at an angle up to about 90°.

14. The device of claim 11, wherein the stopcock is made of a material chosen from at least one of: an acrylic, a polystyrene, a polycarbonate, a wood, and a metal.

15. The device of claim 11, further comprising a tourniquet configured to slide through the enclosure and to receive venapuncture accessories.

16. A method of using a vein stabilizer device, the method comprising the steps of:
threading a tourniquet through a vein stabilizer device, the vein stabilizer device comprising a housing with an aperture therethrough, the aperture configured to slidably receive the tourniquet;
tightening the tourniquet around a patient's arm, thereby distending at least one vein;
selecting the distended vein for puncture after application of the tourniquet and vein stabilizer;
engaging a portion of skin covering the pre-selected vein with a bottom surface of the vein stabilizer device, the vein stabilizer device further comprising a first set of prongs extending from a first edge of the housing, the first set of prongs forming a first groove therebetween, the first groove configured to stabilize a needle and the vein;
stabilizing the distended vein between the grooves; and
puncturing the vein with a needle.

17. The method of claim 16, wherein, in the step of puncturing the vein, the stopcock is held in engagement with the distended vein by the tourniquet.

18. The method of claim 16, further comprising, after the step of puncturing the vein, loosening the tourniquet around the patient's arm, thereby releasing a flow of blood through the vein without disturbing placement of the vein stabilizer device or the needle.

19. The method of claim 16, wherein, in the step of puncturing the vein,
the needle is disposed at angle from the patient's skin, and in a longitudinal direction along the vein; and
wherein both hands of an individual puncturing the vein are disposed behind the needle.

20. The method of claim 16, wherein the vein stabilizer device further comprises a second set of prongs extending from a second edge of the housing, the second set of prongs forming a second groove therebetween, the second groove configured to stabilize the needle and the vein, and wherein the second groove is of a different size than the first groove, and
wherein the step of engaging a portion of skin covering the pre-selected vein with the vein stabilizer device comprises selecting either the first groove or the second groove to engage the vein, based on the size of the groove that is better able to engage both the vein and the needle.

21. The method of claim 16, further comprising the steps of:
after puncturing the vein, rotating one edge of the stopcock upwardly; and
after rotating the edge of the stopcock upwardly, inserting the needle further into the vein.

22. A tourniquet comprising:
an elongated portion configured to receive an accessory for a venapuncture procedure and a vein stabilizer device, wherein the vein stabilizer device comprises
an enclosure with an aperture therethrough, the aperture configured to receive the tourniquet, and
a first set of prongs fixedly extending from a first edge of the enclosure, the first set of prongs forming a first groove therebetween, the first groove configured to receive a needle.

23. The tourniquet of claim 22, wherein the venapuncture procedure accessory is chosen from at least one of the following: vial holder, gauze, bandage, bandage holder, surgical tape, and scissors.
